(19) United States
(12) Patent Application Publication
(10) Pub. No.: US 2024/0009036 A1
(43) Pub. Date: Jan. 11, 2024

(54) PROTECTIVE DEVICE AS A TEMPORARY SEAL DURING SEXUAL ACTIVITIES FOR PREVENTING URINARY TRACT AND BLADDER INFLAMMATION

(71) Applicant: Selina JAKUMEIT, Frankfurt (DE)

(72) Inventor: Selina JAKUMEIT, Frankfurt (DE)

(21) Appl. No.: 18/035,155

(22) PCT Filed: Nov. 5, 2021
(86) PCT No.: PCT/EP2021/080820

§ 371 Date: May 3, 2023

(30) Foreign Application Priority Data
Nov. 6, 2020 (DE) 10 2020 129 363.1

(51) Int. Cl. A61F 13/02; A61F 13/00; A61F 5/00

(52) U.S. Cl. CPC A61F 13/024; A61F 13/00063; A61F 5/0093; A61F 2013/00817; A61F 2013/00604

(57) ABSTRACT

The invention relates to a patch for covering a body site, such as the anus, to a method for applying the patch, and to the use of the patch.

The invention relates to subject matter including protective devices in the form of a temporary seal for bodily orifices and portions of skin, for the purpose of preventing—for example, for minimizing—urinary tract and/or bladder inflammation. The invention relates to patches for covering the anal orifice and the surrounding skin portion, to a method for applying the patch, and to the use of the patch.

Inflammations of the urinary tract and of the bladder (cystitis) are a complaint that is widespread throughout the world. Owing to the anatomy of their short urethra, women are highly susceptible to urinary tract and bladder infections. Sexual activity (sexual intercourse, petting, etc.) is deemed to be a major risk factor and oftentimes trigger for the development of such infections, since in the course of such activity entities including bacteria, such as the individual's intestinal bacteria (for example, Escherichia coli, abbreviated hereinafter to E. coli) and bacteria from the vagina (for example, Gardnerella vaginalis), may pass, as a result of the movement and rubbing, into the closely situated urethra and may trigger inflammation of the urethra and of the bladder. If the infection rises further, this may even lead to pyelonephritis.

A research team from the University of Utah has discovered that certain E. coli bacteria are able to lodge in the bladder wall cells. The E. coli microbes in the cells form so-called biofilms in order to protect themselves, and are therefore protected from attack by drugs and endogenous defense compounds. It has been determined that in 22% of the cases of cystitis triggered by E. coli bacteria, there are bacteria of this kind which have concealed themselves in the cells (page 24, Niedermeier, T. Das Gesundmachbuch—volume 1 (2^{nd} edition 2020). © Das Hemili-Haus Hufnagel/Hufnagel GbR).

It has also been possible to determine, in an experiment on mice, that the bacteria, as soon as they have penetrated into lower-lying cell layers, are able to withstand intact a multi-day antibiotic therapy (page 25, Niedermeier, T. Das Gesundmachbuch—volume 1 (2^{nd} edition 2020). © Das Hemili-Haus Hufnagel/Hufnagel GbR).

Researchers from the Washington University School of Medicine have discovered, in a study on mice, how a bacterium from the vagina is a contributory cause of cystitis. In the study conducted it was discovered that the vaginal bacterium, if it passes into the urethra and into the bladder, has the capacity to damage the epithelium of the bladder. On its own, the bacterium would not trigger any major inflammation in the bladder. The researchers have discovered that because of the damage to the bladder wall, the E. coli bacteria can be released again from their vehicles and so are able to cause cystitis (page 29, Niedermeier, T. Das Gesundmachbuch—volume 1 (2^{nd} edition 2020). © Das Hemili-Haus Hufnagel/Hufnagel GbR).

If, for example, the vaginal bacterium Gardnerella vaginalis enters the urethra/bladder, it may release the intracellular E. coli bacteria from the bladder wall, possibly triggering a renewed cystitis.

The treatment of the urinary tract and bladder infections is frequently lengthy.

In many of the patients, the inflammation develops recurrently and has a considerable adverse effect on quality of life.

In spite of the widespread condition worldwide affecting numerous women, who because of sexual activities suffer repeatedly from urinary tract/bladder infections, the possibilities for protection are confined to preventive household remedies for prevention and to the repressive methods of treatment to control the inflammation once it has arisen.

At present there is no possibility for adopting preventive protection during sexual activities, such as petting or sexual intercourse, for example, against the emergence of the individual's own bacteria from the anus and/or the transfer of bacteria to the surrounding skin portion and/or the penetration of bacteria of any kind into the urethral orifice.

The underlying prior art typically treats the problem with preventive and repressive measures, which often end in the taking of prescription antibiotics.

The preventive measures include the following: adequate consumption of fluids, keeping the feet warm, cotton underwear, the correct wiping technique (“front to back”), passing water after intercourse, the taking of D-mannose, diuretic tea, cranberries, restharrow, horseradish root, nasturtium, inoculation, etc.

If simple household remedies do not help, a visit to a physician is required. Frequently antibiotics or antimycotics are prescribed.

There are a variety of reasons why repeated intake of antibiotics is undesirable. Frequent intake leads to endogenous resistance. Moreover, a treatment with antibiotics combats not only the bacteria triggering the inflammation but also the necessary bacteria which are elemental to the equilibrium of the endogenous immune system. Antibiotic therapies demonstrably destroy the intestinal environment, which is the “headquarters” of our immune system, and their use is therefore not only not helpful but indeed is counterproductive. An ideal breeding ground is created for recurrent urinary tract/bladder inflammation.

Despite the market being furnished with diverse dressing materials, patches, gels having properties for a wide variety of different fields of application, there is presently neither a temporary seal for the anal orifice, nor a temporary seal for the anal orifice and its surrounding skin portion, nor of the urethra, which is designed for the field of application elucidated above. Nor is there any temporary seal which is designed for a different field of application but which could with corresponding reappropriation be utilized readily and safely for the field of application elucidated above.

Conventional skin patches typically feature a classic layer assembly with carrier, adhesive, wound contact material, and liner paper/film.

Blister patches and pressure-stop patches are typically patches with hydrocolloid technology for rapid wound healing.

Herpes patches are customarily patches with hydrocolloid technology for treating herpes. They are used for moist wound treatment.

WO2018133909A1 describes a mucous membrane patch which is produced by electrospinning and is used for the treatment of mucous membrane diseases.

Shower patches are normally water-resistant patches which permit showering.

Probe and dressing fixatives are typically patches for the fastening of probes and dressings.

Tattoo patches are typically patches for covering freshly implemented tattoos.

WO2010138275A2 describes a system in cases of functional disorder of the pelvic nerve and incontinence.

WO2016054292A1 describes a system for assisting with control in cases of incontinence.

WO2006112632A1 describes a gel containing silver for injection into the male urethra in order to avoid infections.

WO2019175055A1 describes a device for administering a gel into the urethra.

WO2014015347A1 and U.S. Pat. No. 8,889,655B2 describe a composition which is applied to the urethral orifice of women.

WO2012094426A2 describes a gel-based closure pad.

EP0288945B1 describes a paste for application to mucous membranes.

U.S. Pat. No. 2,649,854A describes a device for sealing off the urinary outlet.

JP2018007991A describes a pad for sealing off the urinary outlet.

Additionally known, for example, from U.S. Pat. Nos. 5,695,484A, 8,353,884B2, CN208942940U, CN210114575U, CN2671571Y, CN101239015A or US20020192273A1, are a number of patches for closing the anal orifice for the purpose of treating anal incontinence or for the treatment of hemorrhoids.

EP2544643A1 describes a vaginal patch.

The invention is based on the object of providing a technology with which it is possible easily and practicably to prevent, or at least to minimize, the possible emergence of microrobes and bacteria from the anus and/or to prevent or at least significantly hinder the transfer of bacteria and microrobes located on a surrounding skin portion during, for example, sexual acts (petting, sexual intercourse, etc.).

The inventor has recognized that to date there has been no direct or indirect protection against transmission of bacteria into the urethra during sexual intercourse or sexual acts. The invention presented here is intended to offer such protection.

Patch

This object on which the invention is based is achieved, in a first embodiment, by a patch for covering a body site, where said patch
a. has a thickness in a range from 5 to 5000 μm,
b. has a patch carrier layer,
c. has a bonding layer of the patch carrier layer, and
d. has an application layer, which
   i. has a tab into which at least one finger can engage for application of the patch, or
   ii. has a bonding layer of the application layer, to which at least one finger is bonded temporarily for application of the patch, or
   iii. protrudes at least partially beyond the patch carrier layer, where at least the patch carrier layer on as-designated application of the patch is creased and/or bent and/or folded at an angle of 0° to 170°, by virtue of the application layer on as-designated application of the patch being creased and/or bent and/or folded along a center axis of the application layer or cut through along the center axis.
e. has a parting layer of the bonding layer of the patch carrier layer.

The patch is characterized in that the bonding layer of the patch carrier layer in as-designated usage of the patch is configured for full-area adhesion on the body site.

The protruding of the application layer at least partially beyond the patch carrier layer may also be described as follows: The application layer has a first end and a second end, with the first end of the application layer protruding beyond a first border of the patch carrier layer, and with the second end of the application layer protruding beyond a second border of the patch carrier layer, where the patch is embodied in such a way that the application layer and the patch carrier layer can be bent, folded and/or creased along a center axis.

The application layer serves, for example, as a stabilizer and application aid for easy application. For this purpose the application layer possesses, for example, either a tab and/or other adhesion properties to enable temporary securement with or to one and/or two or more fingers.

As a result of the design in accordance with the invention, the patch can be easily applied with a finger, for example, by inserting a finger into the tab or temporarily bonding the finger to the bonding layer of the application layer for the purpose of application. Or by applying the patch for application, creased and/or bent, in the intergluteal cleft with at least two fingers. It is then possible accordingly to locate the patch with the finger easily even in or at sites of the body that are difficult to access.

The patch thus creates a temporary cover with, for example, a flexible and/or tear-resistant, preferably thin patch carrier layer and reliable adhesion, thus allowing the patch to be attached alone without problems, simply and correctly even at sites which are difficult to reach, including those that are outside the field of view, by the application layer of the invention.

The application layer of the invention with the tab can be designed such that the application layer has an application sheet which has one or two or more slots and/or is connected partially and/or over its entire area to the underlying layer such as the patch carrier layer, for example.

The tab may for example have only one opening (1 slit) through which preferably a finger can be inserted. The tab may alternatively have two or more openings (2 or more slits), allowing a finger, for example, to be placed through the tab.

Preferably one of the slits extends with angling at one end up to the border of the application layer. The slit extending up to the border assists the simple removal of the application layer after application to the desired site, since, when the tab is pulled, the slit acts as a kind of predetermined breakage point, which helps to steer the tensile force. If the tab is pulled after application without an extending slit, the tensile force is distributed with relative uniformity in all directions and an unequally greater force is required. As a result of the slit running to the border, the application layer can be removed even more easily after application.

In order to simplify detachment, the parting layer which covers the bonding layer of the patch carrier layer may be provided with a parting line, perforation or the like.

Application by means of application bonding layer: The application layer/application aid of the invention may be designed such that the application layer bonds to its reverse face and is connected partially and/or over the full area, for example, to the underlying patch carrier layer.

The adhesive connection between application layer and patch carrier layer is preferably interrupted at a site on the border. Following application to the desired site, the interruption may be utilized for assistance, in order to enable the application layer to be engaged more effectively from and consequently separated more easily from the patch carrier layer.

After application on the skin, the application layer can preferably be removed or separated off in unipartite form, in other words in one piece.

The patch serves, for example, as a solution for the temporary sealing of the anus and of its surrounding skin portion, in order to prevent, or at least to minimize, the emergence of bacteria from the anus and/or the bacterial transfer of the bacteria on the surrounding skin portion during sexual activities, as quickly, simply, safely, and discreetly as possible.

If the patch is thinner, it may be more difficult to secure the patch for application on a finger either via the tab or the bonding layer of the application layer. If it is thicker, then it may be the case that the patch becomes stiffer and so during application can no longer be attached so easily and so flexibly between buttocks and/or in skin clefts such as, for example, on the anal orifice.

The patch of the invention preferably has an area of 1 cm² to 441 cm², with the patch more particularly having an area of around 20 cm² to 100 cm². If the area of the patch is less than 1 cm², it may become more difficult to secure the patch on a finger for application. In that case, moreover, the patch may no longer be able to provide reliable coverage of bodily orifices such as the anus. If the area of the patch is greater than 441 cm², it may be the case that it can no longer be easily and correctly applied.

The thickness of the patch has a range from 5 to 5000 μm and is situated preferably in a range from 40 to 3000 μm, very preferably in a range from 50 to 2000 μm.

The size and/or shape is preferably designed such as to produce a discreet protection which has no disruptive effects on sexual activities. The shape of the patch and especially the patch carrier layer and its bonding layer is in principle unrestricted. It may, however, be round, angular, triangular, rectangular, square, pentagonal or oval, for example. Other shapes as well, such as rounded corners, for example, are possible.

The size and shape here are preferably chosen such that they reliably cover and close off the desired body site, such as in that case the anus and/or anus and surrounding skin portion, and do not adversely affect or unwantedly cover other sites, such as, for example, the vaginal introitus, clitoris, etc.

The patch carrier layer and/or bonding layer of the patch carrier layer are preferably transparent and/or colorless, in order to be particularly unintrusive and discreet. The term “transparent” in the sense of the invention means that the skin, with the patch in its applied state, can be viewed with the unaided eye through the patch. The transparency is quantified preferably by means of densitometers. Techniques of this kind are familiar to the skilled person. As a measure of the transparency, it is possible with preference to measure the haze as an optical value. The haze is measured preferably according to ASTM test standard D 1003-61 m, Procedure A. In the applied state, preferably at least in one partial region, the patch of the invention has a haze of less than 30%, more preferably less than 25%, more preferably still less than 20%, most preferably less than 15%, and more particularly less than 12%.

“Applied state” in the sense of the invention means that the patch has been applied to the skin and any auxiliary layers of the patch (e.g., parting layers, application layer) are removed.

The patch consists preferably of a plurality of layers connected to one another. The construction of these layers may be, for example, as follows:

Patch Carrier Layer

There may be a patch carrier layer. This layer is the layer which in the applied state is visible, for example, on and around the anal orifice. This layer is, for example, the supporting layer of the patch. The patch carrier layer is preferably highly flexible, so that it holds continuously to planar body sites and curved body portions which are in vigorous motion and does not hinder the action and the freedom of movement of the wearer.

The patch carrier layer may have a thickness in a range from 3 to 300 μm, preferably in a range from 3 to 100 μm. Future materials may offer even thinner layers.

The patch carrier layer may comprise, and preferably consist of, a transparent polymer. The transparent polymer may be a polyurethane or a modified polyurethane. In particular the carrier layer preferably does not consist of rubber or other materials having a greater friction resistance than polyurethane films, in order to reduce rubbing of the outer face of the patch with skin.

The patch carrier layer is preferably transparent, and more preferably is transparent with the levels of haze referred to above in connection with the patch.

The patch carrier layer is preferably uncolored and/or colorless. As a result the patch is also particularly unintrusive on different skin tones.

The patch carrier layer, which in the applied state preferably remains on the body site, preferably does not have an auxiliary tab for the purpose of removal of the patch, for example. As a result of the relative thinness of the patch, it can be removed simply, for example, after utilization, by pressing together of the patch carrier layer and peeling off with two or more fingers.

The patch carrier layer is preferably hypoallergenic. As a result there is particularly little skin irritation especially at sensitive skin portions, such as the anus.

The patch carrier layer preferably has no foam material and/or no cushion. A foam material or cushion would on the one hand be detrimental to the transparency and on the other hand would ensure that the patch could become saturated with fluid and hence could become damp. This also avoids increased friction as a result of the material or of sharp edges.

The patch carrier layer is preferably not absorbent and does not, for example, comprise nonwoven. As a result, the patch is unable to become saturated, and remains thin. In any case provision may be made for the patch or patch carrier layer to comprise neither a nonwoven nor any form of wound contact material. Instead, preferably, the bonding layer of the patch carrier layer comes into contact with the skin over the full area in contact when the patch is applied in the as-designated usage.

The patch carrier layer may also comprise additives such as, for example, antimycotic agents, antibacterial agents, antiviral agents, antiinflammatory agents, antipruritic agents, humic substances, moisture-absorbing agents, gas absorption agents, buffer agents for pH regulation, drying agents, fluid-absorbing materials (for example, hydrophilic agents, such as starch, cellulose, hydrophilic polymers or hydrophilic salts such as, for example, calcium anhydride sulfate), odor-absorbing agents (for example, activated carbon) and/or siccatives (for example, silica gel and the like for the absorption of fluids and odors). Also possible is the addition of, for example, plant-based, nurturing, active medicinal ingredients—for example, addition of vitamins, D-mannose, and/or the 2QR complex.

The reverse face of the patch carrier layer, in other words the side facing the application layer and facing away from the skin, is preferably free from adhesive and/or from adhesion properties.

The patch carrier layer is preferably elastic and has a stiffness which is situated more particularly in a range from 0.01 to 300 N/mm² (2% secant modulus). The stiffness can be measured in accordance with DIN EN ISO 527. If the stiffness is above this range, the wearing of the patch may be very unpleasant and the application be made unnecessary more difficult.

In order to guarantee maximum mobility, the patch has a highly flexible patch carrier layer. The elongation at break of the patch is preferably at least 30%, very preferably at least 60%. The elongation at break can be measured in accordance with standard EN ISO 527. However, it may also be much higher, such as 100-500%, for example.

As a result of the elastic nature of the patch carrier layer and its bonding layer, the patch after use, for example, may be peeled off from the site easily in one piece with two or more fingers, without tearing, for example, in the process.

On account of the thickness and the good bonding force on the skin, and on account of the ultrathin, highly flexible material, for example, there is no risk of injury by rubbing and/or sharp edges.

Patch carrier layer and the bonding layer of the patch carrier layer preferably form a wafer-thin cover which conforms ideally to the body and which is barely perceptible or imperceptible in the state of rest and during movement as well while being worn.

Bonding Layer of the Patch Carrier Layer

With particular preference there is a bonding layer of the patch carrier layer. This layer is the layer which in the applied state is disposed between the patch carrier layer and the skin.

The patch carrier layer and/or its bonding layer preferably have no visible unevenness and/or dishing and/or bumps.

The patch carrier layer and/or the bonding layer of the patch carrier layer preferably has no pores and/or venting holes. As a result it is also possible, for example, for unwanted flatus to be retained. Additionally the risk of permeability for microrobes/bacteria is minimized. With preference it is also possible for a patch carrier layer with a bonding layer to be provided which consist of breathable material, the breathable material being preferably vapor-permeable and fluid-impermeable—but only provided that the impermeability for bacteria and/or microrobes is still ensured. The vapor permeability is advantageous if the patch is to be worn over a relatively long time, as moisture, lubrication and/or perspiration may collect beneath the patch. This may have a negative influence on the adhesion force, but may also give rise to instances of skin irritation or bacterial infection.

The bonding force of the bonding layer of the patch carrier layer is preferably in a range from 2 to 100 N/25 mm, more preferably in the range from 0.05 to 100 N/25 mm.

The bonding force of the layers or of the patch may be measured on steel plates in accordance with standard AFERA 4001 and also analogously on a polyurethane skin model. An alternative assessment of the adhesion force may be accomplished, for example, by way of a test method in accordance with DIN ISO 6133:2017-04 or DIN ISO 6133:2004-05. The bond force may be measured in particular in units of N/25 mm. In the case of the skin model, the bonding force achieved is on average about 2 N/25 mm higher than the average value from testing on human skin.

The bonding force of the patch and/or of the bonding layer of the patch carrier layer may be lower at temperatures of less than 30° C., preferably less than 20° C., and/or at a temperature of above 40° C., preferably of above 45° C., relative to the bonding force at customary body temperatures (34-38° C.). This has the advantage that the patch of the invention may be detached more easily using cold and/or hot water.

With particular preference the bonding layer of the patch carrier layer contains a pressure-sensitive adhesive.

The bonding layer of the patch carrier layer is with particular preference a full-area layer. This has the advantage that a more reliable adhesion is possible for the temporary sealing of the anal orifice and/or a surrounding skin portion, and a comfortable adhesion is more easily possible even in the case of hairs and skin folds.

The bonding layer of the patch carrier layer may be embodied in such a way that the adhesive flows in to the skin folds disposed around the bodily orifice that is to be covered. As a result, the sealing of the bodily orifice with respect to the environment, and hence against the penetration or escape of bacteria, can be promoted. In this way as well the transfer of bacteria and/or microrobes, for example, which are located on the surrounding skin portion, can be hindered by the adhesive masking of the skin portion.

The bonding layer of the patch carrier layer may have a thickness in the range from 5 to 150 μm, preferably in a range from 7 to 50 μm. The bonding layer of the patch carrier layer may be thicker than the patch carrier layer.

The bonding layer of the patch carrier layer may also a thickness in a range from 3 to 150 μm, preferably in a range from 5 to 50 μm.

The bonding layer of the patch carrier layer is preferably transparent and colorless, and more preferably is transparent, with the degrees of haze referred to above in connection with the patch.

The bonding layer of the patch carrier layer is preferably hypoallergenic. As a result there is particularly little skin irritation.

The bonding layer of the patch carrier layer is preferably water-resistant, strongly adhering, and slip-resistant, including preferably on moist substrates and in a moist environment with elevated levels of bodily secretions (for example perspiration, lubrication, etc.).

The bonding layer of the patch carrier layer is preferably detachable from the skin without residue.

The bonding layer of the patch carrier layer may also comprise additives such as, for example, antimycotic agents, antibacterial agents, antiviral agents, antiinflammatory agents, antipruritic agents, humic substances, moisture-absorbing agents, gas absorption agents, buffer agents for pH regulation, drying agents, fluid-absorbing materials (for example, hydrophilic agents, such as starch, cellulose, hydrophilic polymers or hydrophilic salts such as, for example, calcium anhydride sulfate), odor-absorbing agents (for example, activated carbon) and/or siccatives (for example, silica gel and the like for the absorption of fluids and odors). Also possible is the addition of, for example, plant-based, nurturing, active medicinal ingredients—for example, addition of vitamins, D-mannose, and/or the 2QR complex.

Parting Layer

There may be a parting layer. The parting layer is preferably the layer which covers the bonding layer of the patch carrier layer. It serves for example as a covering and protection in order to stave off the drying-out of the bonding layer and/or to keep unwanted soiling or particles away from the bonding layer.

The parting layer may be, for example, a release paper or a release film. For example, the parting layer may be a silicone-coated paper. The paper may preferably have a grammage in a range from 70 to 150 g/m².

In order to simplify the detachment of the parting layer, it may be provided with a parting line, perforation or the like.

Application Layer

The application layer serves for example, as a stabilizer and application aid for easy application, and can be connected to the patch carrier layer through a bonding layer of the application layer. The application layer possesses for example, either a tab or other adhesion properties, to enable temporary fastening on or with one and/or two or more fingers.

The application layer is preferably more stable or stiffer than the patch carrier layer. This stabilizing application layer allows optimal provision of the flexibility of the patch carrier layer optionally with the bonding layer of the patch carrier layer.

The application layer offers stability to the patch until the latter is placed on the skin, and enables reliable positioning even outside the range of view at sites which are difficult to reach such as the anus.

This application layer is preferably removed after application, meaning, for example, that the finger tab disappears and the patch is at its thinnest. Accordingly there are no hindrances during sexual activities, such as getting caught unwantedly on a tab. Nor in that case are there any sharp edges which might otherwise be present because of the tab and/or the border.

The application layer may have a thickness in a range from 5 to 5000 μm, preferably in a range from 15 to 300 μm. The application layer is preferably thicker than the patch carrier layer.

The application layer may comprise polymer film or paper and may consist thereof.

The temporary fastening on or with one and/or two or more fingers may be accomplished, for example, through a tab which is provided by one or more slits. The application layer, accordingly, may have a configuration, for example, such that it has a tab (composed of a slit in the application layer, for example) into which a finger can be placed for application of the patch. The patch may for example have only one opening, through which a finger can be inserted into this tab. The tab may alternatively have two openings, allowing a finger, for example, to be placed through the tab.

In order to simplify the removal of the application layer after it has been applied, in the case of the version with one and/or two or more slits, one of the slits may extend with angling at one of its ends up to the border of the application layer. The slit which runs on one side up to the border of the application layer assists the simple detachment operation, since at this point the application layer can be easily parted from the patch carrier layer by simple pulling and can be detached in one piece, without residues. In this case the slit which runs up to the border acts as a kind of “predetermined breakage point”, which through the pulling of the tab steers the tensile force toward it as the “weakest point”. If the tab is pulled without the slit running through, the tensile force is distributed uniformly from the center and an inequitably greater level of force is required.

Another version of the temporary fastening aid in the form of a tab on the application layer may be designed such that the application layer completely covers the patch carrier layer and, furthermore, has two protruding application strips at two opposite positions, these strips being bonded around the finger.

Another version of the temporary fastening aid in the form of a tab on the application layer may be designed such that an application strip is formed directly on the application layer and can be bonded to it when the finger is placed onto the application layer, so that the finger then lies in a loop made of the application strip.

The temporary fastening for simplified application with one and/or two or more fingers may alternatively be resolved by the application layer, for example, protruding beyond the patch carrier layer, so that it can be guided by one, two or a plurality of fingers to the desired side, such as in this case the intergluteal cleft, without coming into contact with the bonding layer of the patch carrier layer. For this purpose, provision may be made more particularly for the application layer to comprise a first end and a second end, with the first end of the application layer protruding beyond a first border of the patch carrier layer and with the second end of the application layer protruding beyond a second border of the patch carrier layer. The regions or ends of the application layer that protrude beyond the patch carrier layer may in this case be guided or moved toward one another in particular by a user, by creasing or bending of the application layer, or brought together in the case of the cut-through application layer. The temporary fastening on one and/or two or more fingers may alternatively be provided by means of bonding properties such as a bonding layer, for example. Hence there may be an application bonding layer disposed on the application layer. This application bonding layer may serve to allow the patch to be fastened by temporary bonding to one and/or a greater number of fingers, for example.

An application parting layer may be disposed on the application bonding layer. This parting layer prevents unwanted sticking of the patch prior to application, such as within the packaging, for example, and/or prevents the bonding layer drying out and/or prevents unwanted soiling which might possibly adversely affect the bond force. This application parting layer may be removed prior to application, so that the patch can then, for application, be bonded to one and/or a greater number of fingers and fastened thereon, for example. The application parting layer may be, for example, a release paper or a release film. It may for example be a silicone-coated paper. The paper may preferably have a grammage in a range from 70 to 150 g/m².

In order to simplify detachment, it may be provided with a parting line, perforation or the like.

Adhesion Layer of the Application Layer

An adhesion layer of the application layer may be provided, which connects the application layer to the patch carrier layer.

This layer may ensure that, prior to the application of the patch, the application layer adheres on the patch carrier layer and that the application layer can be removed from the patch carrier layer after application. For this purpose the bonding force of the adhesion layer of the application layer may preferably be lower than the bonding force of the bonding layer of the patch carrier layer. This has the advantage that the application layer can be detached more easily from the patch carrier layer after application, without the patch carrier layer parting from the skin with the bonding layer of the patch carrier layer.

The bonding force of the bonding layer of the application layer is preferably in a range from 0.03 to 5 N/25 mm and more particularly in a range from 0.1 to 5 N/25 mm.

The adhesive connection is realized preferably through a bonding layer. The two layers may alternatively be connected to one another by electrostatic adhesion and/or adhesion properties.

Following removal, the bonding layer of the application layer preferably leaves behind no bonding residues on the underlying patch carrier layer.

The bonding layer of the application layer may connect the two layers (patch carrier layer and application layer) to one another at least partially and/or over the full area.

The layer assembly may also comprise more or else fewer than the layers elucidated here. For example, the patch carrier layer and/or the application layer may each be implemented as an assembly of two or more layers, such as two or three layers, for example.

In contradistinction to the situation with fecal incontinence, the patch of the invention is required, for example, merely as a wafer-thin covering for the reliable temporary closure of the anal orifice and/or the anal orifice and its surrounding skin portion, The patch is not required to retain any feces.

The adhesive of the bonding layers and more particularly of the bonding layer of the patch carrier layer is preferably a polyacrylate adhesive. The adhesive may for example comprise polyethylene oxide, polyvinylpyrrolidone, trimethylammonioethyl methacrylate chloride and/or copolymer of ethyl acrylate, methyl methacrylate. The adhesive is preferably hypoallergenic.

The patch is preferably latex-free.

The patch is preferably usable in combination with condoms.

The patch may preferably be utilized during pregnancy.

The patch is preferably usable in combination with lubricating gel.

The patch is preferably usable as a single-use item.

The patch is preferably designed for the human skin.

The patch carrier layer and its bonding layer have an adhesion time of 3 minutes to 3 days, more preferably of 5 minutes to 10 hours, very preferably of 10 minutes to 3 hours.

Method for Applying the Patch

In a further embodiment, the object on which the invention is based is achieved by a method for applying the patch of the invention, wherein the patch is applied on the body by means of the application layer.

The patch is applied preferably on the anus. The patch in this procedure is applied preferably with the application layer and in particular the tab, or with the application bonding layer, or with an application layer which protrudes at least partially beyond the borders of the patch carrier layer. In the configuration of the application layer which protrudes beyond the patch carrier layer, at least the patch carrier layer is preferably bent during application.

The application layer may serve as a stabilizer of the wafer-thin patch and as an application aid and is particularly suitable for use on sites which are difficult to reach, including those lying outside the range of sight of the individual, such as the anus.

A plurality of patches are preferably purchased in a packaging unit.

The patches are preferably packaged individually within the packaging. They may alternatively be arranged in groups of two or more at an offset from one another on the same parting layer. The parting layer is the layer which covers the bonding layer of the patch carrier layer.

The hands and the skin in the genital region, more preferably widely around the anus, including the genital region, are preferably cleaned prior to the application.

Suitable disinfectants and/or cleanses in the form, for example, of disinfecting towels may preferably be included with the patch in order to assist the cleansing prior to the application. Suitable disinfectants are, for example, those suitable for nucleus membranes. Excessively aggressive disinfectants could lead to skin irritation and could dry out the skin.

Application by means of tab: The user takes the patch preferably by both hands at the opposite sides and pushes these sides parallel to the slit/slits in the center direction. The part that is not connected to the underlying patch carrier layer rises up and forms a tab.

The user places preferably one finger in the tab.

The patch is fastened on one finger, and the release layer which covers the bonding layer of the patch carrier layer may be removed, for example, with the other hand, so that the bonding layer of the patch carrier layer is exposed.

Through the fastening of the patch on one finger, for example, the remaining fingers are freely flexible in order to support the correct placement and positioning. For example, by the touching, holding, releasing and/or tightening of the body portion and its near surroundings.

In this way, application is possible even outside the individual's field of view and without mirrors.

When the patch with its bonding layer is down to on, for example, the anus and/or anus and its immediately surrounding skin portion, the user can withdraw the finger from the tab.

The user preferably presses the patch again over the full area after application.

The application layer is preferably removed after application in order to create the optimal wear comfort. With particular preference the application layer is removed in one piece after application.

To remove the application layer, the user can pull on the tab. As a result of the lower adhesive force in comparison to the bonding force of the bonding layer of the patch carrier layer which adheres on and around the anus, the application layer comes away without detaching the patch carrier layer with it.

The application layer may be connected to the patch carrier layer through a bonding layer or by electrostatic charging and/or other adhesion properties. The side of the patch carrier layer that is facing the application layer is preferably free from adhesive or from adhesion properties. The adhesion force of the application layer on the patch carrier layer is more preferably less than the adhesion force of the bonding layer of the patch carrier layer which is applied, for example, on the anus and/or on the anus and its immediately surrounding skin portion. Accordingly, following application, the application layer can be removed without problems, without the patch parting from its substrate or becoming damaged.

The patch can be simply pressed together and peeled off with the assistance, for example, of thumb and index finger.

The patch can be detached preferably without residue.

The patch preferably leaves behind no instances of skin irritation.

The patch may also be used preferably in combination with condoms.

The patch may preferably be used in combination with lubricating gel.

The patch may preferably be utilized during pregnancy as well, owing to its nature.

The patch may also serve to retain unwanted flatus.

After use, the patch can be disclosed of simply and discreetly in the household garbage.

The finger chosen may preferably be the index finger, as this finger is particularly good at placement and in its sense of touch. However, any other finger may also be used.

Application by Means of Application Bonding Layer:

For application, the user preferably removes a parting layer which is covering, for example, the application bonding layer.

In order to make detachment easier, the parting layer may be provided with a parting line, perforation or the like.

The user preferably positions their finger, more preferably one finger, on the bonding application layer.

Through the adhesive fastening of the patch on one finger, for example, the remaining fingers are freely flexible in order to support the correct placement and positioning. For example, by the touching, holding, releasing and/or tightening of the body portion and its near surroundings.

In this way, application is possible even outside the individual's field of view and without mirrors.

When the patch with its bonding layer is mounted on, for example, the anus and/or the anus and its immediately surrounding skin portion, the user can withdraw the finger from the application layer.

The user preferably presses the patch again over the full area after application.

The application layer is preferably removed after application in order to create the optimal wear comfort.

To remove the application layer, the user can pull at the location on the border at which the application layer is not connected to the patch carrier layer.

As a result of the lower adhesive force of the application layer on the patch carrier layer, the application layer can be removed without the patch adhering on and around the anus, for example, being detached with it.

The application of the patch by means of an application layer which protrudes beyond the patch carrier layer may proceed as follows: For the application, the user ensures that at least the patch carrier layer is bent and/or creased along its center axis. The application layer protruding beyond the patch carrier layer is preferably likewise bent and/or creased and/or folded along its center axis. However, the patch may also have been provided with a marking, along which the user can crease, fold and/or bend the patch themselves prior to application. The marking may be a visual marking or an embossment or a perforation or a fold or the like. The application layer protruding beyond the patch carrier layer may, however, also be cut through centrally, so that it stabilizes the bending of the patch carrier layer for application, without itself being bent or creased. The bending comes about, for example, by the user placing the positions that protrude beyond the patch carrier layer onto one another or at least against one another. In this condition, the bonding layer of the patch carrier layer is also creased or bent in such a way that the halves of the bonding layer of the patch carrier layer that result from the crease or the bending project away from one another or point outwardly. Depending on the thickness of the application layer, it may be useful here if the application layer comprises two application layer halves which are separate at a distance from one another in the region of the center axis, allowing them to be folded together.

Preferably with the thumb and the second finger of one hand, the user takes the application layer that protrudes beyond the patch carrier layer and guides it along the crease and/or the bend into the intergluteal cleft. For the application, the user preferably bends forward and/or goes into a crouch and/or otherwise ensures that the cheeks move apart from one another. The use of a reflection agent such as a mirror or a mirrored film, for example, may also be useful. The user can test the correct placement with the other hand, for example, so as not, for example, accidentally to mount the patch too far forward. After the patch has been pressed on the anus and along the intergluteal cleft, a straight posture can be adopted, so that the patch carrier layer with its bonding layer adheres on the skin over the full area and in accordance with the anatomy of the individual, individually and without tensile force or tension. The application layer may then be detached from the patch carrier layer. The at least bent patch carrier layer may be provided by a plurality of embodiments. For example, the application layer may feature a marking along which the user must themselves crease the application layer before it is mounted. In another embodiment, the application layer may already be in a creased form. In another embodiment, the application layer may be cut through centrally, so that a bend in the patch carrier layer results from the ends of the application layer that protrude beyond the patch carrier layer being brought together.

Expressed differently, the application layer may have a first end and a second end, with the first end of the application layer protruding beyond a first border of the patch carrier layer and with the second end of the application layer protruding beyond a second border of the patch carrier layer. In this case the patch is preferably configured in such a way that at least the patch carrier layer during the application of the patch can be bent or creased along a center axis. This center axis runs in particular along a center axis of the patch carrier layer which is equidistant from the first border and from the second border of the patch carrier layer. The ability to be bent or creased in this context means that the patch carrier layer may be bent, creased or folded in such a way that it forms an angle of between 0° to 170°. For application, therefore, the user ensures that at least the patch carrier layer is bent along one of its center axes. The application layer disposed on the patch carrier layer is preferably likewise bent and/or creased and/or folded along one of its center axes.

The patch, accordingly, may also be provided with a marking along which the user has to crease and/or at least fold the patch themselves prior to the application. The marking may be a visual marking or an embossment or a perforation or a fold or the like.

Use of the Patch

In a further embodiment, the object on which the invention is based is achieved through the use of the patch of the invention for covering the anal orifice and/or the anal orifice and its surrounding skin portion.

### FIGURES

The embodiments described above enable the provision of a preventive treatment concept in the form of a protective device for urinary tract and bladder infections that is easy and practicable to utilize preferably during sexual acts in order to prevent, but at least significantly to hinder, the emergence of microbes and bacteria from the anus, for example, and/or the transfer of bacteria/microbes to the surrounding skin portion likewise covered by the patch, in order thus to prevent, or at least to minimize, the risk of urinary tract/bladder inflammation.

Other practical embodiments and advantages of the invention are described below in association with the drawings.

In the drawings:

FIG. 1 shows a five-ply patch 10 having the various layers 12 to 20 of the exemplary embodiment of an application layer with tab. There may be an adhesive layer of the patch carrier layer 18 disposed beneath the patch carrier layer 16. This adhesive layer 18 may be provided with a parting layer 20. Above the patch carrier layer 16 there may be application layer 12 disposed, which is secured on the patch carrier layer 16 with a bonding layer of the application layer 14.

FIG. 2 shows the application layer 12 of the patch 10 in a view from above with one slit (left) 26 and with one slit whose one end may run with angling 28 up to the border (right).

FIG. 3 shows the adhesion layer of the application layer 14, which adheres to the patch carrier layer 16 of the patch 10. The adhesion layer of the application layer may exhibit partial adhesion. The gray regions in FIG. 3 are intended to represent adhesive regions and the white regions are intended to represent non-adhesive regions of the adhesion layer of the application layer 14. The left-hand side shows the exemplary embodiment with two slits in the application layer 12, and the right-hand side shows the exemplary embodiment with one slit in the application layer 12.

FIG. 4 shows the fastening of the patch 10 with a finger 30 in the tab 32. The left-hand side depicts the exemplary embodiment with a slit 26 in the application layer 12, and the right-hand side depicts it with a slit 26 in the application layer 12, one end of said slit running with angling 28 up to the border. FIG. 4 shows a variant in relation to the application of the patch 10. A finger 30 can be inserted into the tab 32, allowing the patch then to be easily positioned on the anal orifice 34 with a finger 30. The tab 32 is preferably part of the application layer 12 or of the patch carrier layer 16.

FIG. 5 shows the application layer 12 of the patch 10 in a view from above with two slits 26 (left) and with two slits 26 of which the end of one slit runs up to the border, 28 (right).

FIG. 6 shows the fastening of the patch 10 with a finger 30 in the tab 32. With two slits 26 on the left-hand side, and on the right-hand side with two slits 26, where one end of one slit runs with angling up to the border, 28. In FIG. 6 the tab 32 has two openings, allowing the finger 30 to be placed through the tab 32.

FIG. 7 shows the application of the patch 10 with a finger 30 in the tab 32 in the variant embodiment with two slits 26, where one end of one slit runs with angling up to the border, 28.

FIG. 8 shows the patch 10 with the additional layers 20 to 24 in a seven-ply construction. There may be a bonding layer of the patch carrier layer 18 disposed beneath the patch carrier layer 16. This bonding layer 18 may be provided with a parting layer 20. Above the patch carrier layer 16 there may be disposed an application layer 12, which is secured on the patch carrier layer 16 with a bonding layer of the application layer 14. On its other side the application layer 12 may have a tacky configuration by means of an application bonding layer 22. And so that this application bonding layer 22 does not stick to other items prior to application, an application parting layer 24 may be disposed on the application bonding layer 22.

FIG. 9 shows the patch 10 in a view from above with application parting layer 12 and possible parting line 44. The parting layer 20 of patch 10 may have the same implementation.

FIG. 10 shows the application bonding layer 22 in a view from above the patch 10. The regions drawn in gray are adhesive and the regions drawn in white are non-adhesive in their configuration—partially adhesive on the left-hand side, adhesive over the full area on the right.

FIG. 11 shows the fastening of the patch 10 with the application bonding layer 22 on a finger 30. The regions drawn in gray are adhesive and the regions drawn in white are non-adhesive in their configuration.

FIG. 12 shows the patch 10 with the application layer 12 and a slit 26. This slit may be widened by the pressing-together of the patch 10, allowing a finger to be placed into the slit 26.

FIG. 13 shows the patch 10 with the application layer 12 and two slits 26. These slits may be widened by the pressing-together of the patch 10, allowing a finger to be placed through between the slits 26.

FIG. 14 shows a specific embodiment wherein the application layer 12 possesses an application strip 48 which is bonded around the finger 30. The application layer 12 is connected to the patch carrier layer 16 with an adhesive layer of the application layer 14.

FIG. 15 likewise shows a specific embodiment, in which an application strip 48 is formed directly on the application layer 12 and can be bonded to it when the finger is placed onto the application layer 12, so that the finger then lies in a loop composed of the application strip 48.

FIG. 16 shows an embodiment of the patch 10 wherein the application layer 12 protrudes beyond the patch carrier layer 16 and the bonding layer of the patch carrier layer 18, and must be creased along the marking 46 for application.

FIG. 17 shows a further embodiment of the patch 10, wherein the application layer 12 protrudes beyond the patch carrier layer 16 and is creased centrally.

FIG. 18 shows an embodiment of the patch 10 wherein the application layer 12 protrudes beyond the patch carrier layer 16 and the bonding layer of the patch carrier layer 18 and is cut through centrally, i.e., consists of at least two parts or application layer halves 12a,b. Depending on the thickness of the application layer 12, the parts or the application layer halves 12a, b may be mounted with a certain distance from one another on the patch carrier layer 16. Hence they can be brought together, for example, without problems, without blocking. The application layer of this embodiment has a first end 52 and a second end 54, with the first end 52 of the application layer 12 protruding beyond a first border 56 of the patch carrier layer and with the second end 54 of the application layer protruding beyond a second border 58 of the patch carrier layer 16. In this case, illustratively, the configuration of the patch 10 is such that at least the patch carrier layer 16 is bendable or creasable along a center axis A during the application of the patch 10. This center axis A runs more particularly along the axis of the patch carrier layer 16 that is equidistant from the first border 56 and from the second border 58 of the patch carrier layer 16. Bendable or creasable in this context means that the patch carrier layer can be bent, creased or folded in such a way that it forms an angle α of between 0° to 170°.

FIGS. 19a,b show embodiments of the patch 10 wherein the patch carrier layer 16 is creased (cf. FIG. 31a) or bent (cf. FIG. 31b). In this creased form the patch carrier layer 16 can be applied without problems to the desired body site, preferably to the anus.

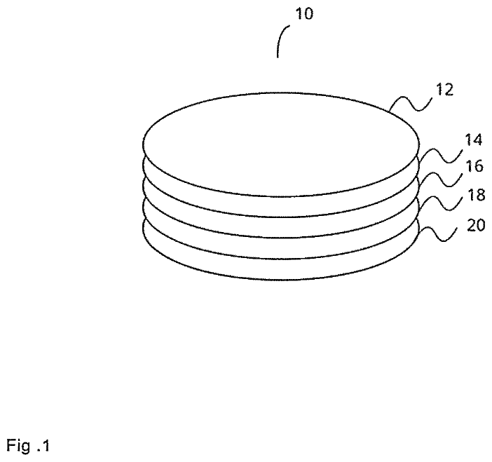

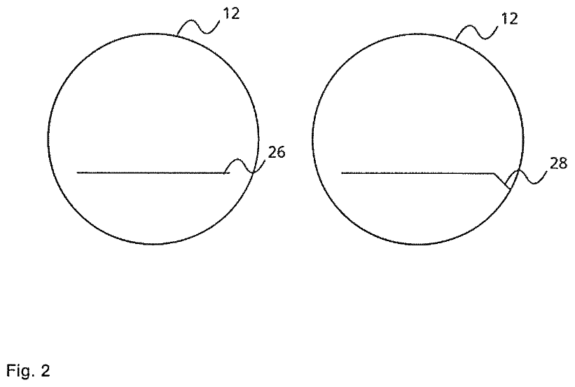

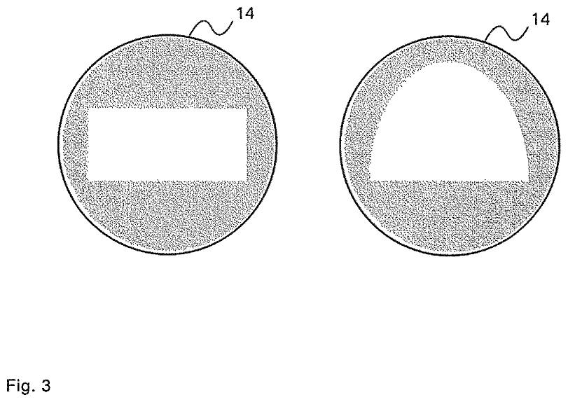

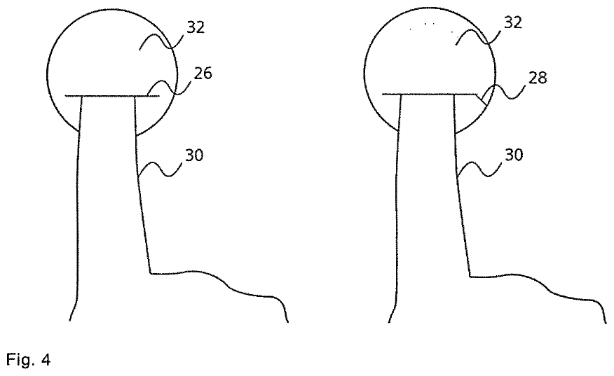

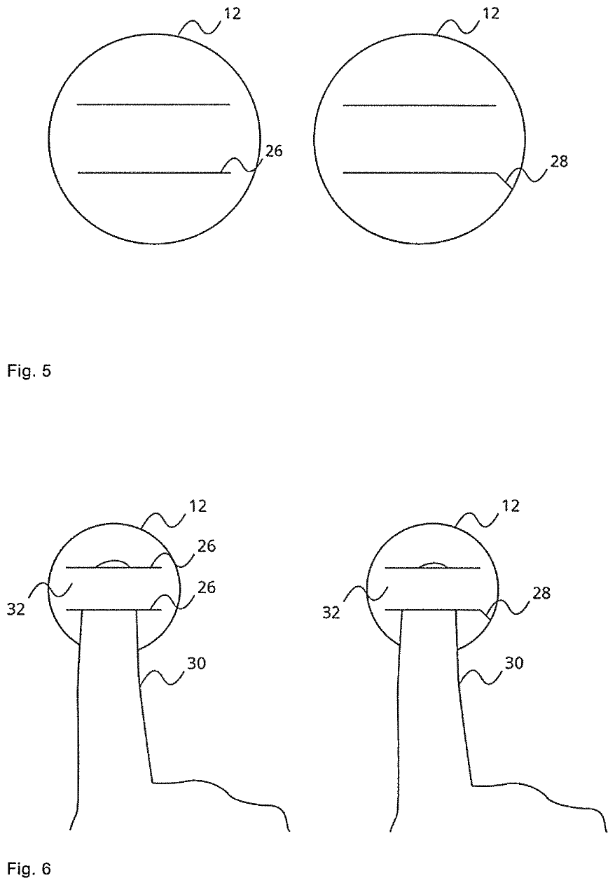

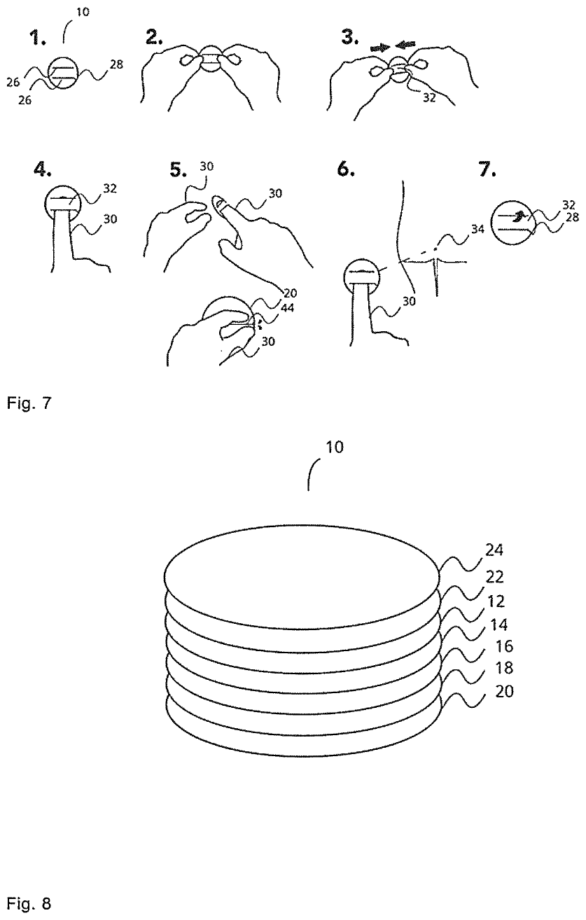

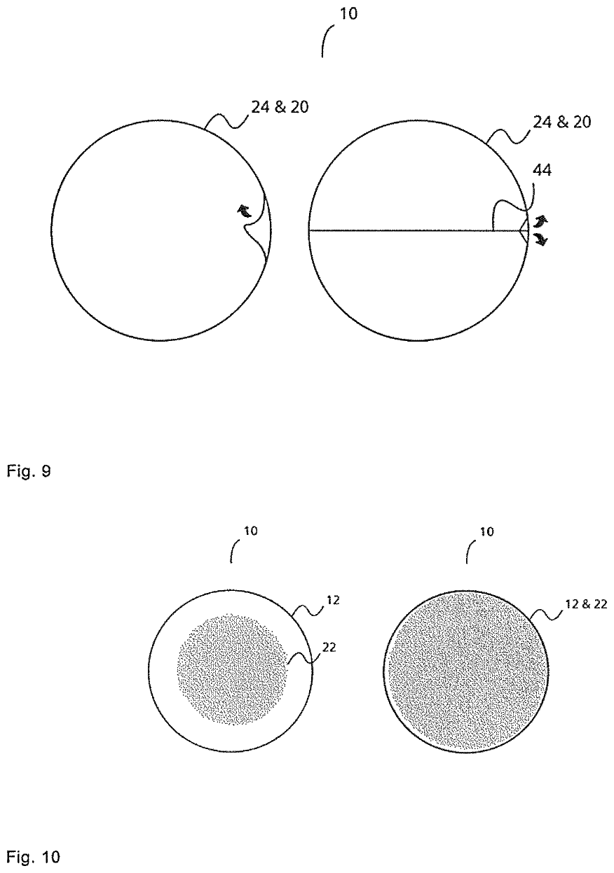

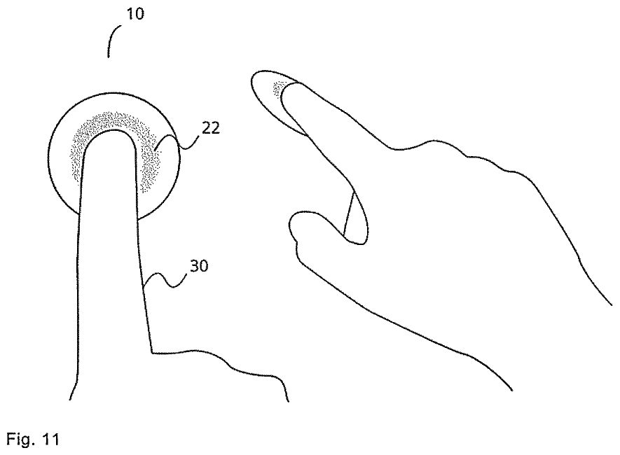

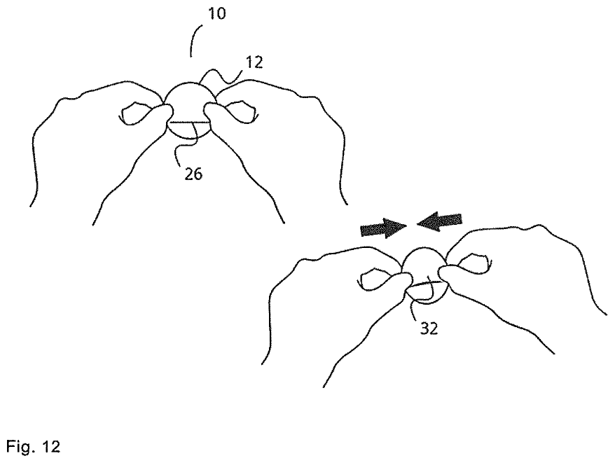

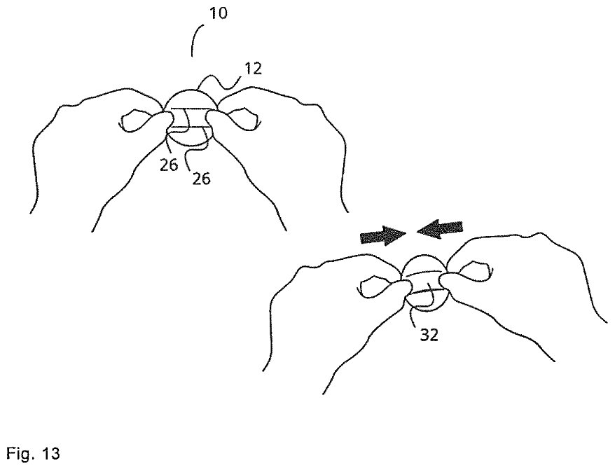

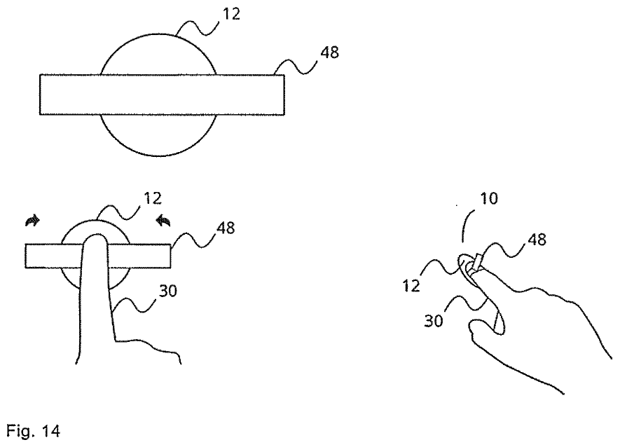

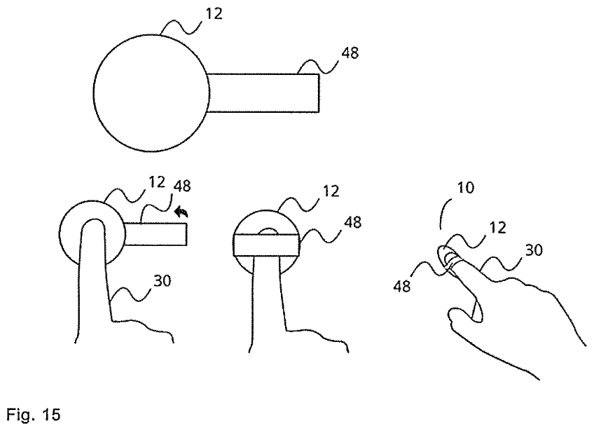

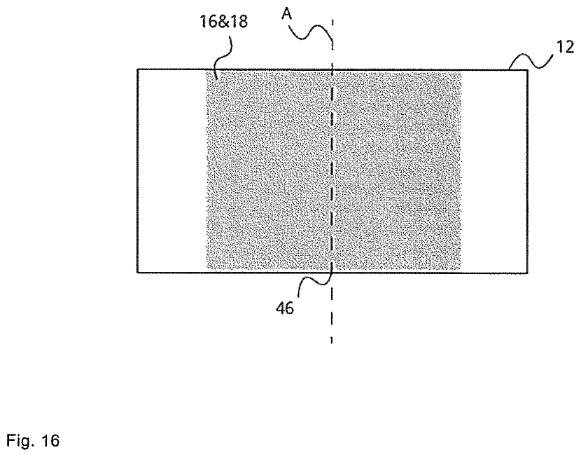

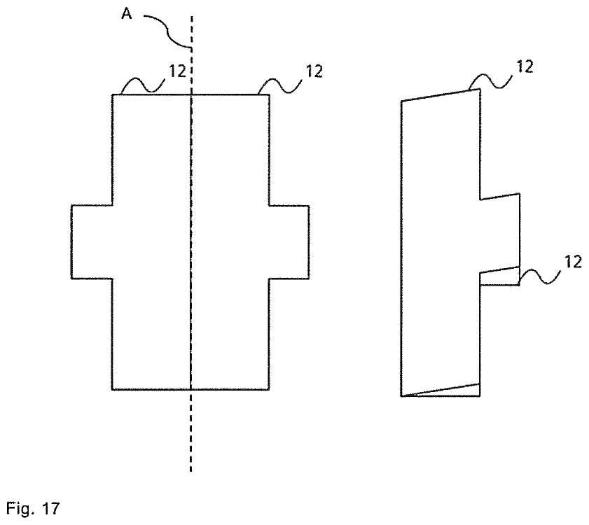

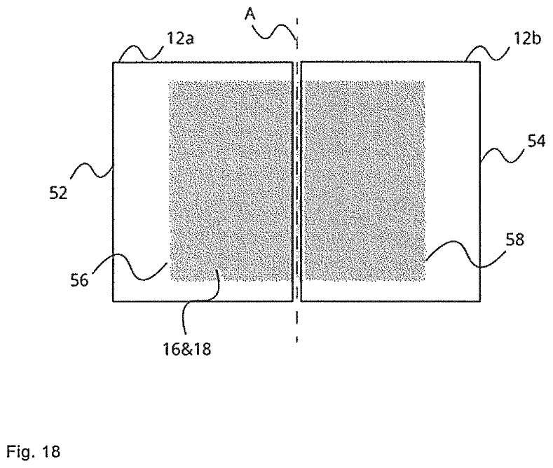

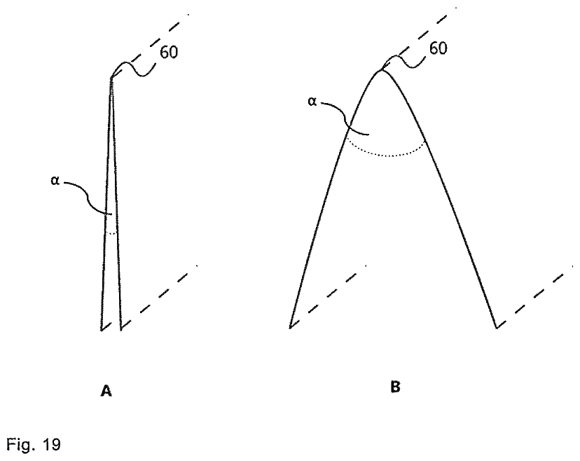

### EXEMPLARY EMBODIMENTS

The invention is illustrated below by specific exemplary embodiments.

### Example 1: Patch

A round patch having a diameter of 5.5 cm was cut from a fastening patch from Augoog Store. For this purpose a circle with a diameter likewise of 5.5 cm was cut from a filing card from Brunnen (grammage 180 g). The circle of paper was provided 2 cm from the lower border with a straight slit having a distance from the border of 1 cm on both sides. On the right-hand side (plane view) the end of the slit was continued to the border at a 45° angle using the cutter.

The border of the paper circle was subsequently coated on its reverse face with the general-purpose adhesive UHU extra from UHU, the coating being such that the adhesive border had a thickness of 1 cm all the way round. The paper circle was then bonded rapidly and flush to the film with the green lines and pressed on firmly.

After a drying time of 30 minutes, the layers bonded to one another were taken so that the paper layer faces upward.

One side of the circle was gripped by the left-hand thumb and index finger at the border, the opposite border of the circle by the right-hand thumb and index finger, so that the slit ran horizontally between the two hands. Then both sides were pressed in the center direction parallel to the slit, causing a tab to form. The index finger of the right hand was positioned into this tab. Subsequently the parting layer on the underside of the circle was detached using the left hand.

The circle was then bonded on the anus with the adhesive, exposed underside. Subsequently, the paper layer, including the film of the fastening patch from Augoog Store, was removed by simple peeling.

This left the wafer-thin film bonded on the anus.

This film was peeled off simply by being pressed together with thumb and index finger, and was disposed of in the household garbage.

The features of the invention as disclosed in the present description, in the drawings, and in the claims may be essential, not only individually but also in any desired combinations, for the actualization of the invention in its various embodiments. The invention is not confined to the embodiments described. It may be varied within the ambit of the claims and with account taken of the knowledge of the competent skilled person.

### LIST OF REFERENCE SYMBOLS

10 patch
12 application layer
12a,b application layer half
14 adhesion layer or bonding layer of the application layer
16 patch carrier layer
18 bonding layer of the patch carrier layer
20 parting layer
22 application bonding layer
24 application parting layer
26 slit
28 angled end of slit
30 finger
32 tab
34 anus
44 parting line
46 marking
48 application strip
52 first end
54 second end
560 first border
58 second border
60 crease
α angle
A center axis

1. A patch (10) for covering a body site, where the patch (10)
a. has a thickness in a range from 5 to 5000 μm,
b. has a patch carrier layer (16),
c. has a bonding layer of the patch carrier layer (18), characterized in that the bonding layer of the patch carrier layer (18) in as-designated usage of the patch (10) is configured for complete adhesion on the body site, and
d. has an application layer (12), which
i. has a tab (32) into which at least one finger (30) can engage for application of the patch (10), or
ii. has a bonding layer of the application layer (22), to which at least one finger (30) is bonded temporarily for application of the patch (10), and an application parting layer (24), or
iii. protrudes at least partially beyond the patch carrier layer (16), where at least the patch carrier layer (16) on as-designated application of the patch (10) is creased and/or bent and/or folded at an angle (α) of 0° to 170° along a center axis (A), by virtue of the application layer (12) on as-designated application of the patch (10) being creased and/or bent and/or folded along the center axis (A) or cut through along the center axis (A) and
e. has an adhesion layer or bonding layer of the application layer (14).

2. The patch (10) as claimed in claim 1, characterized in that the patch carrier layer (16) and/or the bonding layer of the patch carrier layer (18) is transparent.

3. The patch (10) as claimed in claim 1, characterized in that the patch carrier layer (16) has no pores and/or venting holes.

4. The patch (10) as claimed in claim 1, characterized in that the application layer (12) is detachable in one piece from the patch carrier layer (16).

5. The patch (10) as claimed in claim 1, characterized in that the application layer (12) is cut through straight along the center axis (A).

6. The patch (10) as claimed in claim 1, characterized in that the application layer (12) is cut through along the center axis (A) in such a way that two application layer halves (12a,b) are disposed at a distance from one another on the patch carrier layer (16).

7. The patch (10) as claimed in claim 1, characterized in that the application layer (12) is creased and/or bent and/or folded along the center axis (A).

8. The patch (10) as claimed in claim 1, characterized in that the application layer (12) has a marking along the center axis (A).

9. The patch (10) as claimed in claim 1, characterized in that the patch (10) has no nonwoven, no foam material, and no wound pad.

10. The patch (10) as claimed in claim 1, characterized in that the bonding layer of the patch carrier layer (18) and/or the patch carrier layer (16) comprise one or more additives, the additives being selected from the group encompassing antimycotic agents, antibacterial agents, antiviral agents, antiinflammatory agents, antipruritic agents, humic substances, moisture-absorbing agents, gas absorption agents, buffer agents for pH regulation, drying agents, fluid-absorbing materials, hydrophilic agents, starch, cellulose, hydrophilic polymers, hydrophilic salts, calcium anhydride sulfate, odor-absorbing agents, activated carbon, siccatives, silica gel, vitamins, D-mannose, and 2QR complex.

11. The patch (10) as claimed in claim 1, characterized in that the tab (32) is embodied as a slit (26), where the slit (26) is configured with angling at one end (28) up to the border of the application layer (12).

12. A method for applying the patch (10) as claimed in claim 1 to a body site, wherein the patch (10) is applied to the body site by means of the application layer (12).

13. The use of the patch (10) as claimed in claim 1 for covering a body site, such as the anus (34) and/or the anus (34) and its surrounding skin portion, for temporarily preventing the emergence of microbes/bacteria from the anus (34) and/or for preventing the transfer of microbes/bacteria to the skin portions surrounding the anus (34).
